(19) United States
(12) Patent Application Publication
(10) Pub. No.: US 2026/0345841 A1
(43) Pub. Date: Nov. 5, 2026

(54) Intratumoral Alpha-Emitter Radiation in Combination with Immune Checkpoint Regulators

(71) Applicant: ALPHA TAU MEDICAL LTD., Jerusalem (IL)

(72) Inventors: Vered Domankevich, Hod Hasharon (IL); Yona Keisari, Ramat Gan (IL); Itzhak Kelson, Tel Aviv (IL)

(21) Appl. No.: 19/740,362

(22) Filed: Jul. 14, 2026

Related U.S. Application Data
(63) Continuation of application No. 18/569,650, filed on Jun. 19, 2022.
(63) Continuation-in-part of application No. 19/557,204, filed on Mar. 5, 2026.
Application No. 19/557,204 is a continuation of application No. 17/288,926, filed on Oct. 31, 2019, now Pat. No. 12,594,291.
(60) Provisional application No. 62/753,930, filed on Nov. 1, 2018.
(60) Provisional application No. 63/212,671, filed on Jun. 20, 2021.

(51) Int. Cl. A61K 51/12; A61K 9/00; A61K 31/4245; A61K 31/437; A61K 31/454; A61K 31/4985; A61K 31/635; A61K 39/00; A61K 39/395; A61P 35/00

(52) U.S. Cl. CPC A61K 51/1282; A61K 9/0024; A61K 31/4245; A61K 31/437; A61K 31/454; A61K 31/4985; A61K 31/635; A61K 39/3955; A61P 35/00; A61K 2039/505

(57) ABSTRACT

A method of treating a patient with a tumor including treating the tumor with intra-tumoral alpha-emitter radiotherapy and administering, to the patient, a checkpoint inhibitor. In some cases, the substance is administered at least 3 days after beginning the treating of the tumor with intra-tumoral alpha-emitter radiotherapy. The checkpoint inhibitor includes, for example, an anti-LAG3 checkpoint inhibitor, an anti-PD-1 checkpoint inhibitor, an anti-PDL-1 checkpoint inhibitor, an anti-CTLA4 checkpoint inhibitor, an anti-TIGIT checkpoint inhibitor and/or an anti-TIM-3 checkpoint inhibitor.

### CROSS-REFERENCE TO RELATED APPLICATIONS

The present application is a continuation of U.S. patent application Ser. No. 18/569,650, filed Dec. 13, 2023, which is a national phase of PCT application PCT/IB2022/055680, published as PCT publication WO 2022/269446, filed Jun. 19, 2022. This PCT application claims the benefit under 35 U.S.C. § 119(e) of U.S. provisional application 63/212,671, filed Jun. 20, 2021.

The present application is also a continuation in part of U.S. patent application Ser. No. 19/557,204, filed Mar. 5, 2026, which is a continuation of U.S. patent application Ser. No. 17/288,926, filed Apr. 27, 2021, now U.S. Pat. No. 12,594,291, which is a national phase of PCT application PCT/IB2019/059331, published as PCT publication WO 2020/089819, filed Oct. 31, 2019. This PCT application claims the benefit under 35 U.S.C. § 119 (e) of U.S. provisional application 62/753,930, filed Nov. 1, 2018.

### FIELD OF THE INVENTION

The present invention relates generally to tumor therapy and particularly to combined intra-tumoral alpha-emitter radiation and immune checkpoint regulators.

### BACKGROUND OF THE INVENTION

Cancer is the primary cause of death in many countries around the world. Accordingly, an enormous volume of resources has been spent on treatments for cancer, and a wide variety of such treatments have been suggested.

One class of tumor therapy is tumor ablation, to kill tumor cells in situ. In addition to killing cells in situ, tumor ablation may induce an anti-tumoral immune response resulting in the elimination of residual and distant tumor cells. This happens due to the dispersion of tumoral antigens and danger signals that are released from dead and/or dying tumor cells. Tumoral antigens are captured by antigen-presenting cells (APCs), such as dendritic cells (DCs), that in turn present those to T-cells, for example, via a cross presentation pathway as described in Nakayama, Masafumi. “Antigen presentation by MHC-dressed cells.” Frontiers in immunology 5 (2015): 672.

Multiple ablation methods have been proposed, such as, high or low temperature, microwave, laser, electric, photodynamic, chemical (e.g., using reactive oxygen species (ROS)) and radioactive ablation, which could be applied externally (e.g., external beam radiation therapy) or internally (e.g., brachytherapy), and can include different types of radiation such as alpha radiation, beta radiation and photon radiation. A discussion of these methods appears, for example, in Keisari, Yona. “Tumor abolition and antitumor immunostimulation by physico-chemical tumor ablation.” Front Biosci 22 (2017): 310-347. The ablation method used for any specific patient is generally selected according to the type of the tumor, its location size, stage and/or other parameters of the tumor.

Another class of tumor therapy, referred to as immunotherapy, involves the enhancement of a patient's immune response against tumor cells. Many immunotherapy methods have been suggested, such as: immune checkpoint inhibitors, Toll-like receptor (TLR) agonists (e.g. CpG), local gene therapy, cytokine therapy, antibodies against certain protein targets, CAR-T cell therapy, dendritic cell vaccine, adoptive transfer of tumor infiltrating lymphocytes, inhibition of immune suppressor cells, and oncolytic virotherapy. These methods are discussed, for example, in Papaioannou, Nikos E., et al. “Harnessing the immune system to improve cancer therapy.” Annals of translational medicine 4.14 (2016). Generally, the specific method used for each patient is selected according to the type of the tumor or its stage. Multiple combinations of the above discussed therapy types were tested in pre-clinical and clinical trials, as described, for example, in Table 1 in Aznar, M. Angela, et al. “Intratumoral delivery of immunotherapy act locally, think globally.” The Journal of Immunology 198.1 (2017): 31-39. In the case of treatment with immune checkpoint inhibitors, for example, response rates to the treatment are relatively low (about 20%). Patients that receive the treatment mostly do not respond, yet develop serious adverse effects. An extensive effort is made to find treatments that may enhance response rates to immune checkpoint inhibitors, currently, without a pronounced success.

A paper of Sean McBride et al. titled “Randomized Phase-II Trial of Nivolumab with Stereotactic Body Radiotherapy Versus Nivolumab Alone in Metastactic Head and Neck Squamous Cell Carcinoma”, Journal of Clinical Ocology, vol. 39, issue 1, pages 30-38, describes a trial which found no improvement from addition of stereotactic body radiotherapy to nivolumab.

### SUMMARY OF THE INVENTION

An aspect of some embodiments of the invention relates to tumor treatment based on a synergy between immune checkpoint regulators and intra-tumoral alpha-emitter radiotherapy. The term intra-tumoral refers herein to a treatment in which alpha-emitter radionuclides are implanted on a seed within a tumor in one or more initial locations and they or their daughter radionuclides travel to other locations in the tumor, at which alpha-emitting decay occurs. The travel of the radionuclides from the seed may be due to diffusion or due to decay, when the radionuclides on the seed begin a chain of a plurality of radioactive decays.

The various options and alternatives listed in the following description and claims may be used in the alternative or together in any suitable combination, except where the options are specifically contradictory.

There is therefore provided in accordance with an embodiment of the present invention, a substance which regulates immune-checkpoints for use as a medicament for treatment of a tumor of a patient wherein the administration pattern of the medicament comprises administering a therapeutically effective amount of the substance to the tumor, in one or more sessions, and implanting seeds carrying radium-224 in the tumor for intra-tumoral alpha-emitter radiotherapy less than two weeks from administering the substance.

Optionally, the administration pattern of the medicament comprises beginning the administering the substance within less than five days from implanting of the seeds. Optionally, the administration pattern of the medicament comprises beginning the administering of the substance at least 12 hours after the implanting of the seeds. Optionally, the administration pattern of the medicament comprises beginning the administering of the substance at least 72 hours after the implanting of the seeds. Optionally, the administration pattern of the medicament comprises beginning the administering of the substance at least 12 hours before the implanting of the seeds. Optionally, the administration pattern of the medicament comprises beginning the administering of the substance at least 72 hours before the implanting of the seeds. Optionally, the substance comprises an antiangiogenic agent. Optionally, the substance comprises a checkpoint inhibitor. Optionally, the substance comprises small molecule inhibitors. Optionally, the substance comprises Nivolumab, pembrolizumab, cemiplimab, toripalimab, or sintilimab. Optionally, the substance comprises Atezolizumab, avelumab, or durvalumab. In some embodiments, the substance comprises Ipilimumab, Relatlimab, LY3321367, Tiragolumab, Epacadostat and/or Enoblituzumab.

Optionally, the substance comprises HLX23 or ORIC-533. Alternatively or additionally, the substance comprises Monalizumab, pexidartinib and/or Lacnotuzumab. Optionally, wherein the substance comprises Pepinemab. Optionally, the substance comprises Enapotamab. Optionally, the substance comprises tavolimab or cudarolimab. Optionally, the substance comprises Vopratelimab, sotigalimab or Elotuzumab. In some embodiments, the seeds comprise a support having a length of at least 1 millimeter; and radium-224 atoms coupled to the support such that not more than 20% of the radium-224 atoms leave the support into the tumor in 24 hours, without decay, when the seed is implanted in the tumor, but upon decay, at least 5% of daughter radionuclides of the radium-224 atoms leave the support upon decay.

There is further provided in accordance with an embodiment of the present invention, a method of treating a patient with a tumor, comprising treating the tumor with intra-tumoral alpha-emitter radiotherapy; and administering, to the patient, a substance which regulates immune checkpoints within two weeks of beginning the treating of the tumor with intra-tumoral alpha-emitter radiotherapy.

Optionally, administering the substance comprises administering an immune checkpoint inhibitor and/or an immune checkpoint bi-specific antibody. In some embodiments, administering the substance comprises administering a molecule that internalizes immune checkpoints. In some embodiments, administering the substance comprises administering a LAG3 checkpoint inhibitor. In some embodiments, administering the substance comprises administering a PD-1 checkpoint inhibitor. In some embodiments, administering the substance comprises administering a PDL-1 checkpoint inhibitor. In some embodiments, administering the substance comprises administering a CTLA4 checkpoint inhibitor.

In some embodiments, administering the substance comprises administering a small molecule inhibitor. In some embodiments, administering the substance comprises administering a Costimulatory molecule. In some embodiments, administering the substance comprises administering nivolumab or pembrolizumab. In some embodiments, administering the substance comprises administering atezolizumab, avelumab or durvalumab. In some embodiments, administering the substance comprises administering ipilimumab or Tremelimumab. In some embodiments, administering the substance comprises administering Relatlimab. In some embodiments, administering the substance comprises administering tebotelimab. In some embodiments, administering the substance comprises administering TSR-022. In some embodiments, administering the substance comprises administering Etigilimab or Tiragolumab. In some embodiments, administering the substance comprises administering Enoblituzumab, pomalidomide, berzosertib and/or celecoxib. In some embodiments, administering the substance comprises administering Vemurafenib. In some embodiments, administering the substance comprises administering vorinostat. In some embodiments, administering the substance comprises administering sorafenib or sunitinib. In some embodiments, administering the substance comprises administering tavolimab. In some embodiments, administering the substance comprises administering Elotuzumab. In some embodiments, administering the substance comprises administering the substance at least 72 hours after beginning the treating of the tumor with intra-tumoral alpha-emitter radiotherapy. In some embodiments, administering the substance comprises administering the substance less than two weeks after beginning the treating of the tumor with intra-tumoral alpha-emitter radiotherapy. In some embodiments, administering the substance comprises administering the substance less than 144 hours after beginning the treating of the tumor with intra-tumoral alpha-emitter radiotherapy. In some embodiments, administering the substance comprises administering an immune checkpoint blockade.

There is further provided in accordance with an embodiment of the present invention, a kit for treatment of a patient, comprising: at least one source for being at least partially introduced into a body of a subject, having alpha-emitting atoms mounted thereon, at least one immune checkpoint regulator; and a package containing the at least one source and the at least one immune checkpoint regulator.

There is further provided in accordance with an embodiment of the present invention, an alpha-emitting device designed for use in alpha-emitter radiotherapy treatment of a tumor of a patient wherein the alpha-emitter radiotherapy treatment pattern comprises treating the tumor with the alpha-emitter device followed by administering a therapeutically effective amount of an immune checkpoint regulator, in one or more sessions, less than six weeks after beginning the alpha-emitter radiotherapy. Optionally, the alpha-emitter radiotherapy treatment pattern comprises treating the tumor with the alpha-emitter device followed by administering a therapeutically effective amount of an immune checkpoint regulator, in one or more sessions, less than two weeks after beginning the alpha-emitter radiotherapy.

There is further provided in accordance with an embodiment of the present invention, an alpha-emitting device designed for use in alpha-emitter radiotherapy treatment of a population having a tumor treated with a therapeutically effective amount of an immune checkpoint regulator, in one or more sessions, less than six weeks after beginning the alpha-emitter radiotherapy.

Optionally, the device comprises a support having a length of at least 1 millimeter; and radium-224 atoms coupled to the support such that not more than 20% of the radium-224 atoms leave the support into the tumor in 24 hours, without decay, when the device is implanted in the tumor, but upon decay, at least 5% of daughter radionuclides of the radium-224 atoms leave the support upon decay.

### BRIEF DESCRIPTION OF THE DRAWINGS

FIG. 1 is a flowchart of a therapy method, in accordance with an embodiment of the invention;

FIG. 2 is a schematic illustration of a kit for combined alpha-emitter radiation and immune checkpoint regulators, in accordance with an embodiment of the invention;

FIG. 3 is a graph showing results of an experiment testing an effect of a combined alpha-emitter radiation and anti-PD-1 on mouse squamous cell carcinoma tumor development, in accordance with an embodiment of the invention;

FIG. 4 is a graph showing results of an experiment testing an effect of a combined alpha-emitter radiation and anti-PD-1 on mouse pancreatic tumor development, in accordance with an embodiment of the invention;

FIG. 5 is a graph showing results of an experiment testing an effect of alpha-emitter radiation on the activation of dendritic (DC) cells in mouse squamous cell carcinoma mice tumors;

FIGS. 6A-6C are dot plots showing results of an experiment testing an effect of a combined alpha-emitter radiation and anti-PD-1 on CD3+, CD8+ and Granzyme B T-cells, respectively, in mouse squamous cell carcinoma mice tumors, in accordance with an embodiment of the invention;

FIG. 6D shows CD3 T-cell density in squamous cell carcinoma tumor tissue following treatment with a-PD1 versus DaRT together with a-PD1, in accordance with embodiments of the present invention; and

FIG. 7 is a graph showing results of an experiment testing an effect of a combined alpha-emitter radiation and anti-PD-1 on immune myeloid derived suppressive cells (MDSCs) in the mouse spleen, in accordance with an embodiment of the invention.

### DETAILED DESCRIPTION OF EMBODIMENTS

An aspect of some embodiments of the invention relates to a combined tumor treatment including alpha-emitter radiation in a tumor of a patient and applying a therapeutically effective treatment of one or more immune checkpoint regulators to the patient within a time frame which achieves a synergy between the alpha-emitter radiation and the one or more immune checkpoint regulators. In some embodiments, the one or more immune checkpoint regulators are administered to the patient within 4 weeks or even within two weeks, before or after, the beginning of the alpha-emitter radiation treatment. Applicant has found that the combination of the specific tumor ablation of applying alpha-emitter radiation followed by the specific immunotherapy of immune checkpoint regulators has a substantially greater therapeutic effect than each of the treatments separately. While traditional radiation therapy can damage the natural immune system of the patient for example by harming immune organs or by expanding immune suppressive cells such as MDSCs and therefore may counteract the benefits of immune checkpoint regulators, applicant found that alpha-emitter radiation has a positive effect when applied with immune checkpoint regulators, while reducing the immune suppressive populations.

According to experiments done by the applicant, following alpha-emitter radiation, dendritic cells (DC) are activated in the tumor within about 1 week following alpha-emitter radiation. According to this finding, applicant has determined that starting the immune checkpoint regulator treatment within two weeks from beginning the alpha-emitter radiation treatment may be advantageous. In addition, applicant has shown that under this sequencing between treatments, the combined treatment leads to a synergy in T-cells infiltration to the tumor, which is a well-documented criterion for the responsiveness to immune checkpoint regulators. In addition, applicant has found that T cell function that is expressed by granzyme B secretion, is elevated only in the combination treatment. Additionally, systemic MDSCs populations are reduced in the combinational treatment relative to aPD-1 monotherapy.

It is noted that in applicant's experiments applicant found that seven days after beginning the DaRT treatment there was a mass destruction of cells in the tumor, including T-cells. In addition, at this stage, cells that performed negative regulation entered the tumor. After another 7 days, however, in days 14-16 from the beginning the DaRT treatment, there was an unexpected increase in T-cells and the functioning of the T-cells. Additionally, at that stage, cells that performed negative regulation showed a decrease. This unexpected change did not occur when treating only with immune checkpoint regulators.

It is believed by applicant that alpha-emitter radiation primes effector T-cells to respond to checkpoint regulation by enabling the coexistence, over space and time, of immune cells in the tumor microenvironment with dead/dying cells following alpha-emitter insertion. Intra-tumoral Diffusing alpha-emitter Radiation Therapy (DaRT) utilizes alpha emitting atoms for the treatment of tumors, optionally by employing a source coated with radioactive atoms. The alpha emitting atoms are released locally, in a controlled-release manner, both in time and in space. Namely, the atoms spread in the tumor gradually. At an initial timepoint most of the radioactivity is concentrated near the source. As time passes, this distribution changes in a way that some of the atoms migrate from the location close to the source to a more distant location in the tumor. In addition to the changes of the migration distance of the radioactive atoms with time, for each timepoint there is a different distribution of activity as a function of the distance from the source. Namely, for each given timepoint and migration distance there is a different amount of activity along the line between the source and the migration maximal distance, where points closer to the source generally have a higher activity. This enables a non-unified and non-immediate destruction of the tumor tissue.

Immune checkpoint regulators, particularly immune checkpoint inhibitors, inhibit the negative regulation on cytotoxic T-cells, allowing them to properly recognize and kill tumor cells. For a T-cell to identify a tumor cell, an event of activation should first occur, which is dependent on the interaction between the T-cell and an activated antigen presenting cell (APC). This interaction may happen in the lymph nodes or in the tumor itself. Generally, immune checkpoint inhibitors will not function if no T-cell clone that was previously activated by an APC to recognize a specific tumor antigen exists. In case the T-cell was previously activated by an APC specifically towards a tumor antigen, T lymphocytes may infiltrate into the tumor that presents this antigen, and this may condition a successful action of the immune checkpoint inhibitor. Such a process may also occur for metastatic cells.

Tumor cell killing by DaRT leads to the induction of a specific anti-tumor immune response. This process involves a local inflammatory response, recruitment of APCs such as dendritic cells and macrophages, and their activation by tumor antigens released from dead cells or presented on dying cells, and by Damage-Associated Molecular Patterns (DAMPs), eat-me signals and cytokines present in the tumor microenvironment. The antigen loaded and activated APCs present the tumor antigens to the T-cell for its specific activation. It is believed by the applicant that since DaRT-induced DNA damage is considered complex and the release of the radioactive atoms from the seed is gradual, the DaRT mediated in-situ tumor destruction and ensued inflammation result in a stronger and long lasting systemic and specific adaptive immune response against a wide range of tumor antigens. Moreover, the applicant found that DaRT reduces the abundance of suppressive immune cells that compromise T-cell function such as MDSCs. In contrast to other tumor ablation therapies, DaRT does not invoke a complete destruction of the tumor microenvironment immediately but rather gradually. This may allow the co-existence of APCs and T-cells with dying/dead cells, enabling the interactions required to activate cytotoxic T-cells such that their function will then be enhanced with checkpoint inhibitors. In addition, due to the short-ranged effect of DaRT, important immune organs such as lymph nodes and bone marrow, and tertiary lymphoid structures in the close vicinity of the tumor, remain intact, to support the local and systemic immune response.

### Treatment Method

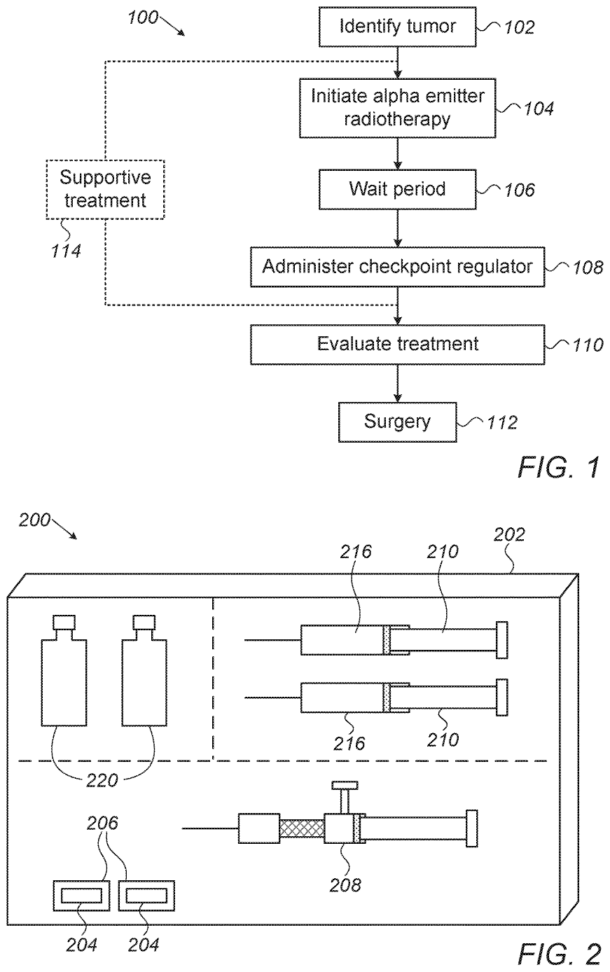

FIG. 1 is a flowchart of a therapy method 100, in accordance with an embodiment of the invention. Further to identification (102) of a tumor in a patient, therapy method 100 begins with initiating (104) an alpha-emitter radiation treatment (referred to herein also as alpha-emitter radiotherapy), for example by implanting of alpha-emitter radiation sources into the tumor. A limited time period (106) after initiating the alpha-emitting radiation treatment, a therapeutically effective immune checkpoint regulator is administered (108) to the patient in one or more sessions.

In some embodiments, after the alpha-emitter radiation treatment is completed, the effect of the treatment is evaluated (110). In some embodiments, following evaluation, a surgery (112) to remove the residual primary tumor is employed. In some embodiments, the surgery is carried out at least a week or even at least 14 days following the beginning or completion of the radiation treatment. While surgery to remove a cancerous tumor is generally performed as soon as possible, applicant has found that after applying a combined immune checkpoint regulation and alpha-emitter radiotherapy treatment it is better to wait in order to allow the treatment to take effect and only then to remove the tumor. Alternatively, surgery is performed at any other suitable time, possibly before the alpha-emitter radiotherapy, or is not performed at all, when deemed unnecessary or unfeasible. Further alternatively or additionally, the evaluation (110) is not performed. In some embodiments, therapy method 100 further includes providing (114) a supportive treatment.

### Tumor Types

Therapy method 100 may be used in treatment of any tumor type, including cancerous tumors, benign neoplasms, in situ neoplasms (pre-malignant), malignant neoplasms (cancer), and neoplasms of uncertain or unknown behavior. In some embodiments, the method of FIG. 1 is used to treat relatively solid tumors, such as breast, kidney, pancreatic, skin, head and neck, colorectal, ovarian, bladder, brain, vulvar and prostate cancer. In other embodiments, the method of FIG. 1 is used to treat non-solid tumors. The method of FIG. 1 may be used for both primary and secondary tumors.

Exemplary tumors that can be treated by the method of FIG. 1 include but are not exclusive to tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer; breast cancer, susceptibility to breast cancer, type 4 breast cancer, breast cancer-1, breast cancer-3; breast-ovarian cancer), squamous cell carcinoma (e.g., in head and neck), vulvar cancer, neurogenic tumor, astrocytoma, ganglioblastoma, neuroblastoma, gliomas, adenocarcinoma, adrenal tumor, hereditary adrenocortical carcinoma, brain malignancy (tumor), various other carcinomas (e.g., bronchogenic large cell, ductal, epidermoid, large cell, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, solid lymphoma, liposarcoma, lynch cancer family syndrome II, male germ cell tumor, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

In some embodiments, the method of FIG. 1 is applied to a tumor known to be affected substantially by alpha-emitter radiation on its own. In other embodiments, the method of FIG. 1 is applied to a tumor of a type which is not affected substantially by alpha-emitter radiation on its own, for example does not reduce in size at all or does not reduce in size by more than 5% or 10%.

Experiments performed by applicant indicate that even tumors that do not show enhanced T-cell infiltration to the tumor following alpha-emitter radiation treatment or immune checkpoint regulator treatments on their own, enhance dramatically T-cell infiltration into the tumor when targeted by the combination of immune checkpoint regulators and alpha-emitter radiation in accordance with the method of FIG. 1. In addition, this T-cell infiltration is in correlation to reduction in tumor size, supporting a functional t-cell response.

### Immune Checkpoint Regulators

The immune checkpoint regulators may be small molecules, antibodies (also known as blockades) or any other types of drugs which effect the regulation of immune checkpoint pathways.

In some embodiments, the immune checkpoint regulators comprise immune checkpoint inhibitors, which inhibit the function of one or more molecules of cells, such as immune checkpoint molecules. Immune check point inhibitors can be antibodies or small molecules. Table 1 lists various molecules which may be targeted by the immune checkpoint inhibitors along with specific corresponding immune checkpoint inhibitors.

TABLE 1

| Inhibited protein | Possible checkpoint inhibitors |
|---|---|
| PD-1 | Nivolumab, pembrolizumab, cemiplimab, toripalimab, sintilimab, |
| | SB415286, dostarlimab. |
| PDL-1 | Atezolizumab, avelumab, durvalumab, Envafolimab, BMS- |
| | 936559, cosibelimab, CS-1001, SHR-1316, CBT-502, BGB- |
| | A333, MPDL3280A, BMS-8, BMS-37, BMS-202, BMS-230, |
| | BMS-242, BMS-1001 and BMS-1166 |
| CTLA4 | Ipilimumab, tremelimumab, compounds “1-6” |
| Lag3 | Eftilagimod alpha, Relatlimab, LAG525, MK-4280, Sym022, |
| | IMP321, REGN3767 (R3767), BI 754,091, tebotelimab |
| | (MGD013), FS118, TSR-033, BMS-986016 (BMS-ONO), |
| | GSK2831781, GSK503 |
| Tim 3 | Sym023, TSR-022, MBG453, INCAGN2390, LY3321367, BMS- |
| | 986258, RO7121661, BI754111, Sym022, MGD013, FS118, |
| | SHR-1702, RMT3-23, ATIK2a, EPZ005687, DZNep, IBI110 |
| TIGIT | Etigilimab, Tiragolumab, BMS-986207, AB-154, ASP-8374, |
| | OMP-313M32, MK-7684 |
| IDO (Indoleamine-pyrrole | Epacadostat, navoximod, IO102-IO103 |
| 2,3-dioxygenase) | |
| VISTA | JNJ-61610588, CA-170, CI-8993, SNS-101, |
| B7-H3 or B7-H4 | Enoblituzumab, MGA271, MGA217, I-Omburtamab, I-8H9, |
| | MGC018, DS-7300a, Orlotamab, MGC018, FPA150, MGD009 |
| BTLA | TAB004 |
| Siglec-15 | NC318 |
| A2aR | EOS100850, AB928 |
| CD73 | HLX23 CPI-006, ORIC-533 |
| NKG2A | Monalizumab |
| PVRIG/PVRL2 | COM701 |
| CEACAM1 | CM24 |
| CEACAM 5/6 | NEO-201 |
| CCL2/CCR2 | PF-04136309 |
| LIF | MSC-1 |
| CD47/SIRPα | Hu5F9-G4 (5F9), ALX148, TTI-662, RRx-001, KWAR23 |
| CSF-1 (M-CSF)/CSF-1R | Lacnotuzumab (MCS110), LY3022855, SNDX-6352, |
| | emactuzumab (RG7155), pexidartinib (PLX3397) |
| IL-1 and IL-1R3 (IL- | CAN04, Canakinumab (ACZ885) |
| 1RAP) | |
| IL-8 | BMS-986253 |
| SEMA4D | Pepinemab (VX15/2503) |
| Ang-2 | Trebananib |
| CLEVER-1 | FP-1305 |
| Axl | Enapotamab vedotin (EnaV) |
| Phosphatidylserine | Bavituximab |

In other embodiments, the immune checkpoint regulators comprise immune checkpoint bi-specific antibodies, such as any of the following:
a. anti-CTLA-4+OX40 (ATOR-1015)
b. anti-PDL1+Lag3 (MGD013, FS118)
c. anti-PDL1+TGF beta receptor (M7824)
d. anti-PDL1+TIGIT
e. anti PDL1+4-1BB (INBRX-105, ATG-101)
f. anti pd1+CTLA4 (MGD019)
g. anti PD-1+TIM-3 (RO7121661)
h. anti CD47-PD-L1 (PF-07257876)
i. anti PD-L1+Claudin18.2 (Q-1802)
j. ImmTACs (IMC-F106C, IMCgp100-tebentafusp)
k. Anti PD-1+LAG3 (RO7247669, Tebotelimab)
l. Anti pd-1+CD137 (IBI319)
m. Anti CD137+HER-2 (PRS-343)
n. Anti PD-1+ICOS (IZURALIMAB)
o. Anti PD-L1+CD27 (CDX-527)

In still other embodiments, the immune checkpoint regulators comprise molecules that inhibit immune checkpoint expression, such as any of the following:
a. ATR inhibitors (e.g., berzosertib)
b. Cox-2 inhibitors (e.g., celecoxib)
c. BRAF inhibitors (e.g., Vemurafenib, Dabrafenib, Encorafenib)
d. MEK inhibitors (e.g., trametinib, binimetinib, selumetinib, and cobimetinib)
e. PI3K inhibitor (e.g., Idelalisib, Alpelisib Taselisib, Pictilisib, Duvelisib, Copanlisib, Gedatolisib, Apitolisib, Dactolisib)
f. HDAC inhibitors (entinistat, vorinostat, Mocetinostat, Panobinostat, ACY-241)
g. DNMT inhibitors (decitabine, guadecitabine, Azacitidine)
h. Bromodomain inhibitors (JQ1, I-BET151)
i. RTK inhibitors (Cediranib, semaxinib)

In still other embodiments, immune checkpoint regulators comprise molecules that inhibit immune checkpoint expression and in addition are antiangiogenic agents, which prevent development of blood vessels, such as:
a. IMiDs (e.g., pomalidomide, lenalidomide, brefelamide, thalidomide, iberdomide, apremilast)
b. TK inhibitors (sorafenib, dasatinib, sunitinib, nilotinib, gefitinib, eriotinib, bosutinib, lapatinib, pazopanib, regorafenib, Lestaurtinib, Imatinib)

In other embodiments, the immune checkpoint regulators combined with alpha-emitter radiation comprise molecules that internalize immune checkpoints. Such molecules may include, for example, ARB-272572, and/or ARB-276309.

In some embodiments, the immune checkpoint regulators comprise immune costimulatory molecules. Since these molecules act on immune pathways related to those affected by immune checkpoint inhibitors, the applicant concludes that using these molecules with DaRT will lead to similar results due to positive regulation on these pathways via stimulation rather than via removal of negative regulation, such as one or more of the following:
A) OX40 (tavolimab, cudarolimab, GSK3174998, DB36, DB71, DB15, CVN, MGCD0103, SNDX-275, INBRX-106, PF-0451860)
B) ICOS (GSK3359609, JTX-2011/Vopratelimab, MEDI-570, KY104
C) CD137/4-1BB (PF-05082566)
D) SLAM (Elotuzumab)
E) CD40 (APX005M (sotigalimab), SEA-CD40, CDX-1140, MP0317)
The administered immune checkpoint regulator may include a single drug, or may include a combination of a plurality of different drugs of the above, administered together or in separate sessions.

### Route of Administration

In some embodiments, the administering of the immune checkpoint regulators to the tumor and/or to metastases is done by systemic administration, for example orally or by intravenous (IV) injection or infusion, In some embodiments, the delivery of the immune checkpoint regulator uses a suitable method of targeted delivery.

Alternatively or additionally, the immune checkpoint regulator is administered (108) in situ, directly to one or more identified tumors. Optionally, in this alternative the immune checkpoint regulator is administered by intra-tumoral injection. While in some embodiments the immune checkpoint regulators are administered from the seeds which carry the alpha-emitter radionuclides, preferably the immune checkpoint regulators are administered separately from the seeds, in order to achieve a wider coverage of the tumor which receives and is affected by the immune checkpoint regulators.

Optionally, before administering the immune checkpoint regulators to a patient, a size of the tumor, tumors and/or metastases is estimated and accordingly an amount of the immune checkpoint regulators to be administered is selected.

### Timing

In some embodiments, the checkpoint regulator is administered (108) to the patient, in a single session. Alternatively, immune checkpoint regulators are administered (108) in multiple sessions, possibly at least three, at least seven or even at least twelve sessions. The separate sessions are optionally separated by at least four hours, eight hours, 24 hours, 48 hours or even at least 72 hours, from each other. The multiple sessions optionally use the same immune checkpoint regulator. Alternatively, different sessions use different immune checkpoint regulators. For embodiments in which the immune checkpoint regulators are administered (108) in multiple sessions, the following paragraphs relate to the first session of administration, unless stated otherwise.

One embodiment found to provide particularly promising results included a first immune checkpoint inhibitor dose session 1-2 days after alpha-emitter radiotherapy source insertion and continuing the treatment for about two weeks.

In a first class of embodiments, the timing of the immune checkpoint regulators therapy is selected so that at first, alpha-emitter radiation is applied without the immune checkpoint regulators, so that T-cells are not induced by the immune checkpoint regulators to infiltrate into the destruction area of the alpha-emitter radiation, when the alpha-emitter radiation is most potent.

Optionally, in this first class of embodiments, the administering of the immune checkpoint regulators begins a limited buffering time period (106) after implanting the alpha-emitting sources (104). The buffering time period is optionally selected to have the immune checkpoint regulators take effect, after a given percentage of the alpha-emitter particles on the seeds undergo decay. The given percentage, is optionally at least 10%, at least 20%, at least 30% or even at least 50%.

The buffering time period (106) between the alpha-emitter radiotherapy induction (104) and initiating (108) the immune checkpoint regulation treatment, is selected to allow the alpha-emitter radiotherapy induction to take effect before administering the immune checkpoint regulator. For example, when the alpha-emitter radiotherapy induction is induced by the insertion of alpha emitter source, the limited time period (106) is selected to serve as a buffering period suitable to allow upregulation of MHC1 expression on the tumor cells' membrane, cytokines and DAMPs secretion and activation of APCs, due to the specific type of killing effect of alpha-emitter radiotherapy on tumor cells. Alternatively, or additionally, the length of the time period (106) is selected as sufficient in order to allow time for the killed tumor cells to activate immune cells.

The buffering time period (106) between implanting (104) the alpha emitter sources and the first session of administering (108) the immune checkpoint regulators is optionally at least 6 hours, at least 9 hours, at least 12 hours, at least 24 hours, at least 48 hours, at least 96 hours or even at least 120 hours. Optionally, the buffering time period (106) is shorter than one month, shorter than three weeks, shorter than two weeks, shorter than 10 days, shorter than a week, shorter than 120 hours, shorter than 96 hours, shorter than 72 hours or even shorter than 48 hours, so that the effects of the alpha-emitter radiotherapy already activated immune cells when the immune checkpoint inhibitor is applied. Optionally, the buffering time is sufficiently short so that the enhanced infiltration of T-cells into the tumor takes effect before the tumor has a chance to recover and reproduce malignant cells in a large scale. In some embodiments, the limited time period (106) is shorter than 30 hours or even shorter than 20 hours, for example in tumors which react more quickly to the alpha-emitter radiotherapy.

In a second class of embodiments, the immune checkpoint regulators are administered after most of the radionuclides on the implanted seed underwent radioactive decay, which occurs within about two weeks.

In a third class of embodiments, the immune checkpoint regulators are administered before the implanting of the alpha-emitting seeds, so that the immune checkpoint regulators operate substantially throughout the duration of the radiotherapy. This class of embodiments is used, for example, in particularly malignant types of tumors, where it is best to begin attacking the malignant cells as soon as possible, without a delay due to waiting for the alpha-emitter radiotherapy seeds. The immune checkpoint regulators are optionally administered in this class of embodiments, at least 6 hours, at least 12 hours, at least 24 hours or even at least 48 hours, at least 72 hours, at least 96 hours or even at least a week before the beginning of the alpha-emitter radiation treatment. Alternatively, the immune checkpoint regulators are administered a short time before the implanting of the alpha-emitting seeds. For example, the immune checkpoint regulators are administered in accordance with this alternative less than 72 hours, less than 48 hours, less than 24 hours, less than 12 hours or even less than 6 hours before the implanting of the seeds.

In some embodiments, after implantation and/or activation of alpha-emitter seeds, one or more parameters of the tumor are monitored in order to determine a time point most suitable for applying the immune checkpoint regulators therapy. The monitoring optionally includes imaging the tumor, using a suitable modality (e.g., X-ray, ultrasound, PET-CT, MRI, CT) to identify when the tumor begins to change due to the activation of alpha-emitter radiotherapy. Alternatively, the monitoring includes performing a blood test to identify levels of an attribute. It is noted, however, that in some embodiments the immune checkpoint regulators are administered before the effect of the alpha-emitter radiotherapy is detectable.

### Alpha-Emitter Radiation

The alpha-emitter radiation treatment is optionally implemented by insertion of seeds carrying radioactive atoms such as Radium-224 or Radium-223, which release alpha emitting atoms inside the tumor. Optionally, the alpha emitting atoms are attached to the seed in a manner that the atoms do not leave the seed, but upon radionuclide decay resulting daughter radionuclides leave the seed. The seed optionally emits daughter radionuclide atoms at a rate of at least 0.1%, 0.5% or even at least 1% of the number of radionuclide atoms coupled to the seed when originally employed, per 24 hours. In some embodiments, the daughter radionuclide atoms are slowly released from the seed, at a rate of less than 25%, less than 10%, less than 5% or even less than 3% of the radionuclide atoms coupled to the seed, per 24 hours.

Alternatively to attaching the alpha emitting atoms to the seed in a manner that the atoms do not leave the seed without radionuclide decay, the alpha emitting atoms are attached to the seed in a manner that the atoms controllably leave the seed at a rate of at least 0.1% per 24 hours, in methods other than radionuclide decay, as described, for example in PCT publication WO 2019/193464, titled “controlled release of radionuclides”, which is incorporated herein by reference.

In some embodiments, the alpha-emitter radiation comprises diffusing alpha-emitter radiation therapy (DaRT). The alpha-emitter radiotherapy is optionally carried out using any of the methods and/or devices described in U.S. Pat. No. 8,834,837, US patent publication 2009/0136422, U.S. provisional application 62/913,184, filed Oct. 10, 2019, and/or PCT publication WO 2018/207105, which are incorporated herein by reference.

The alpha-emitter radiation treatment is optionally initiated (104) by inserting one or more seeds comprising alpha emitting atoms on an outer surface of the seeds, into the tumor. Alternatively, the alpha-emitter radiotherapy is initiated by activating previously inserted seeds, carrying alpha emitting atoms. Optionally, in accordance with this alternative, the seeds are inserted into the patient with a bio-absorbable coating, which prevents alpha-radiation and/or the daughter radionuclides from leaving the seeds. The bio-absorbable coating optionally comprises polylactide (PLA), polyglycolide (PGA) or co-polymers of PLA and PGA, tailored to achieve a desired resorption rate of the coating. Alternatively, or additionally, the coating comprises co-poly lactic acid/glycolic acid (PLGA). The polymers of the coating optionally have molecular weights ranging from 5,000 to 100,000. The material of the coating dissolves in the patient through any of the methods known in the art, such as one or more of ultrasonic energy, reaction with body temperature and/or reaction with body fluids. Additional discussion of bio-absorbable polymers which may be used in accordance with embodiments of the present invention after adjustment for the desired resorption rate are described in U.S. Pat. No. 8,821,364 and US patent publication 2002/0055667, which are incorporated herein by reference. In some embodiments, the initiation (108) includes applying a stimulus which dissolves the coating and thus allows the alpha radiation and/or the daughter radionuclides to leave the seed. In other embodiments, the initiation (108) is achieved by the dissolving of the coating due to contact with tissue of the tumor, without further physician initiation.

The alpha-emitter radiotherapy is optionally applied to the patient for at least 24 hours, at least 5 days, at least 10 days or even at least 14 days. Spreading the destruction of the tumor cells over such a period allows time for the immune checkpoint regulator to help the patient's immune system to accommodate to the changes and participate in destroying remains of the tumor and/or metastases. In some embodiments, the seed is removed from the patient after a designated treatment duration. For example, the seed is optionally removed during surgery for removal of the tumor. Alternatively, the seed is not removed. In some embodiments, the seed comprises a biodegradable material.

### Additional Treatment

Providing (114) the supportive treatment comprises, in some embodiments, one or more treatments which counter undesired side effects of the radiotherapy and/or of the immune checkpoint inhibitor. Optionally, the supportive treatment comprises one or more treatments that counter accelerated tissue repair induced by the alpha-emitter radiotherapy, as such accelerated tissue repair will support residual tumor cells and promote tumor recurrence. Alternatively or additionally, the supportive treatment comprises one or more anti-inflammation treatments which downregulate inflammation following tissue damage caused by the radiotherapy and/or the immune checkpoint inhibitor. In some embodiments, the supportive treatment comprises one or more treatments which prevent DNA repair, so as to interfere with attempts of the body of the patient to repair DNA of tumor cells damaged by the radiotherapy. In other embodiments, the supportive treatment comprises one or more treatments which stimulate a pathogen attack.

In some embodiment, the supportive treatment comprises one or more immunostimulators, such as immunoadjuvants, cytokines, RIG-1 agonists, STING-agonists and/or TLR agonists.

In some embodiment, the supportive treatment includes a treatment which has two or even three of the above listed effects.

In some embodiments, the supportive treatment comprises a supportive immuno-modulation, for example, any of the treatments known in the art for inhibition of immuno-suppressor cells, such as myeloid-derived suppressor cells (MDSCs) and/or Tregs inhibitors (e.g., Cyclophosphamide) and/or activation of TLR pathway (TLR agonists). The MDSCs inhibitors include, for example, indoleamine 2,3-dioxygenase 1 (IDO1) inhibitors, such as Epacadodtat, TGFb inhibitors, such as Galunisertib, PDE5 inhibitors, such as Sildenafil and/or Cox 2 inhibitors, such as etodolac.

Alternatively, or additionally, the supportive treatment administering one or more pattern recognition receptors and/or agonists, such as TLR7,8 (e.g., MEDI9197, Imiquimod), TLR9 (e.g., MGN1703, SD-101, TLR4, GSK1795091, G100, GLA-SE), TLR3 (e.g., Poly-ICLC) and/or STING (e.g., MIW815).

Alternatively, or additionally, the additional treatment comprises administering DNA repair inhibitors of a type found to increase, not to affect or to only minimally impede the immune responses induced by alpha-emitter radiotherapy. In some embodiments, the administered DNA repair inhibitors include ATR inhibitors, for example berzosertib, AZD6738, and/or NU6027. Alternatively, or additionally, the DNA repair inhibitors include ATM/ATR inhibitors, such as KU-55933, KU-60019 and/or EPT-46464, DNA-PK inhibitors (e.g., 6-Nitroveratraldehyde, NU7441), Weel inhibitors (e.g., adavosertib), Hsp90 inhibitors (e.g., Tanespimycin) and/or PARP inhibitors (e.g., Olaparib, Talazoparib).

Alternatively, or additionally, the additional treatment comprises anti-angiogenic factors of a type found to increase, not to affect or to only minimally impede the immune responses induced by alpha-emitter radiotherapy and/or the immune checkpoint inhibition. In some embodiments, the additional treatment comprises Bevacizumab or iMiDs for example Pomalidomide, Thalidomide, Lenalidomide and/or Apremilast.

Further alternatively or additionally, the additional treatment comprises local or systemic chemotherapy treatment, of a type found not to interfere with the alpha radiation and/or the immune responses to immune checkpoint inhibition and/or the alpha-emitter radiation. Optionally, the chemotherapy treatment comprises one or more of Cyclophosphamide (CP), doxorubicin, gemcitabine, oxaliplatin and/or cisplatin.

In some embodiments, the additional treatment comprises alternatively or additionally, anti-inflammatory drugs, such as NSAIDs, e.g., Cox2 inhibitors.

Further alternatively or additionally, the additional treatment comprises administering one or more epigenetic drugs, such as DNMT inhibitors (e.g., Decitabine, Azacytidine, Guadecitabine) and/or HDAC inhibitors (e.g., Entinostat, vorinostat).

In some embodiments, the additional treatment is provided (114) while the alpha-emitter radiation is applied. In other embodiments, the additional treatment is provided (114) after the alpha-emitter radiotherapy is completed, for example after the majority of the radionuclides in the seeds underwent a nuclear reaction, and/or after the seeds are removed from the patient. In still other embodiments, the additional treatment is provided (114) before the alpha-emitter radiotherapy.

In some embodiments, the supportive treatment is provided within less than 72 hours, less than 48 hours or even less than 32 hours from one of the immune checkpoint regulation sessions and/or the radiotherapy treatment.

The timing of providing the additional treatment is optionally selected according to the specific type of the additional treatment.

Optionally, the additional treatment is provided (114) responsively to the tumor type.

### Treatment Kit

FIG. 2 is a schematic illustration of a kit 200 for treatment of a patient in accordance with the method of FIG. 1. Kit 200 comprises a sterile package 202 including one or more alpha-emitter radiotherapy seeds 204, for insertion into a tumor, and one or more doses 216 of an agent/agents for immune checkpoint inhibition.

Optionally, the seeds 204 are provided within a vial or other casing 206 which prevents radiation from exiting the casing. In some embodiments, the casing is filled with a viscous liquid, such as glycerine, which prevents radiation from escaping the casing 206, such as described in PCT application PCT/IB2019/051834, titled “Radiotherapy Seeds and Applicators”, the disclosure of which is incorporated herein by reference. In some embodiments, kit 200 further includes a seed applicator 208, which is used to introduce seeds 204 into the patient, as described in PCT application PCT/IB2019/051834. Optionally, applicator 208 is provided preloaded with one or more seeds 204 therein. In accordance with this option, separate seeds 204 in casings 206 are supplied for cases in which more than the number of preloaded seeds is required. Alternatively, seeds 204 in casings 206 are not provided in kit 200 and only seeds within applicator 208 are included in the kit 200.

As shown, the doses 216 of the immune checkpoint inhibitor are provided preloaded in one or more needles 210. In other embodiments, the doses 216 are provided in one or more containers or vials and the needles are provided separately within sterile package 202 or are not provided in kit 200, at all.

In some embodiments, kit 200 further includes one or more drugs 220 required for the supportive immuno-modulatory treatments (114).

In some embodiments, kit 200 includes a plurality of separate compartments, separated by suitable insulation, for substances which require storage at different temperatures. For example, a first compartment may include dry ice which keeps the substances in the first compartment at about −20 °C, while a second compartment includes ice which keeps the substances in the second compartment at about 4 °C.

The radiotherapy seeds 204 optionally comprise a metallic or non-metallic support, which is configured for insertion into a body of a subject. The seeds 204 further comprise radionuclide atoms of e.g., radium-224, on an outer surface, as described, for example, in U.S. Pat. No. 8,894,969, which is incorporated herein by reference. The radionuclide atoms are generally coupled to the seed in a manner such that radionuclide atoms do not leave the support, but upon radioactive decay, their daughter radionuclides may leave the seed 204 due to recoil resulting from the decay. The percentage of daughter radionuclides that leave the support due to decay is referred to as the desorption probability. The coupling of radiotherapy atoms to the seed is achieved, in some embodiments, by heat treatment. Alternatively or additionally, a coating covers the seed and atoms, in a manner which prevents release of the radionuclide atoms, and/or regulates a rate of release of daughter radionuclides, upon radioactive decay. Daughter radionuclides may pass through the coating and out of the seed 204 due to recoil or the recoil may bring them into the coating, from which they leave by diffusion.

Seed 204 comprises, in some embodiments, a seed for complete implant within a tumor of a patient, and may have any suitable shape, such as a rod or plate. Alternatively to being fully implanted, seed 204 is only partially implanted within a patient and is part of a needle, a wire, a tip of an endoscope, a tip of a laparoscope, or any other suitable probe.

In some embodiments, seed 204 is cylindrical and has a length of at least 1 millimeter, at least 2 millimeters, or even at least 5 millimeters. Optionally, the seeds 204 have a length of between 5-60 mm (millimeters). Seed 204 optionally has a diameter of 0.7-1 mm, although in some cases, sources of larger or smaller diameters are used. Particularly, for treatment layouts of small spacings, seed 204 optionally has a diameter of less than 0.7 mm, less than 0.5 mm, less than 0.4 mm or even not more than 0.3 mm.

### Experiments

FIG. 3 shows the results of an experiment applicant performed to test the method of FIG. 1. In the experiment, Balb/c mice bearing SCC tumors were treated as follows. aPD-1 group received an inert source plus mouse anti-PD-1 intraperitoneally on days 2, 6, 9, 13 in the dose of 10 mg/kg. DaRT group received a 6.5 mm DaRT seed loaded with 75 kBq Ra-224 on day 0 plus control antibody. Inert (control) group received and inert source plus control antibody. DaRT+aPD-1 group received a 6.5 mm DaRT seed loaded with 75 kBq Ra-224 on day 0 plus anti-PD1 intraperitoneally on days 2, 6, 9, 13 in the dose of 10 mg/kg. No effect on tumor development was observed for aPD-1 treatment compared to control. DaRT significantly reduced tumor development compared to control. The combinational treatment inhibited tumor development compared to control and to DaRT groups. This indicates that beyond the obvious killing effects of DaRT due to the speared of alpha emitting atoms, in the presence of checkpoint blockade, another type of killing (probably T-cell mediated) affects tumor size. This suggests that DaRT activates the immune system towards response to checkpoint blockade. This experiment was repeated (aPD-1 doses were given on days 2, 4, 8, 12) with similar results.

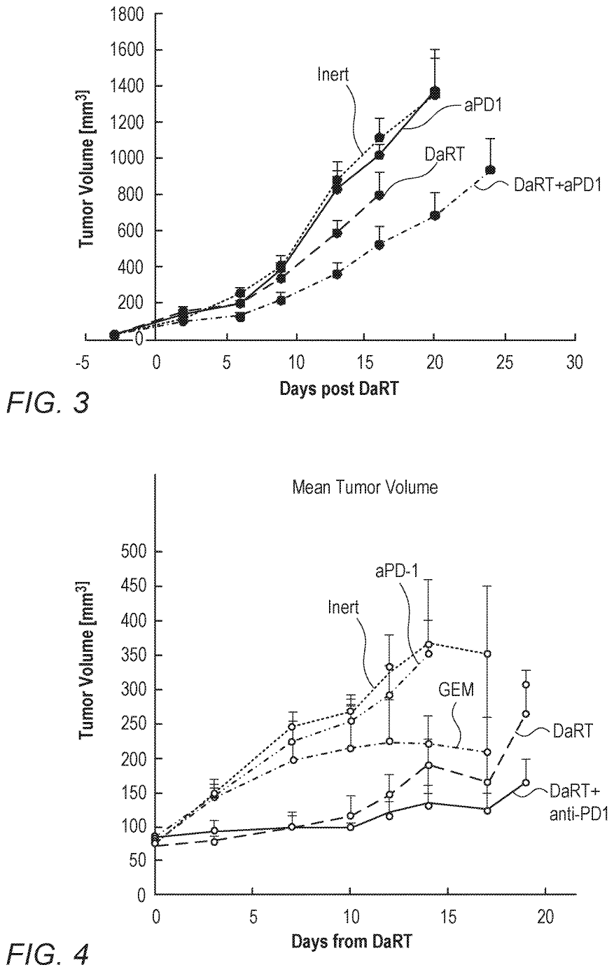

FIG. 4 shows the results of an experiment applicant performed to test the method of FIG. 1. In the experiment, C57BL/6 micebearing Pancreatic ductal adenocarcinoma (PDAC) tumors were treated as follows: aPD-1 group received an inert source plus mouse anti-PD-1 intraperitoneally administered on days 1, 4, 7, 10, 14 in the dose of 10 mg/kg. DaRT group received a 6.5 mm DaRT seed loaded with 80 kBq Ra-224 on day 0 plus control antibody. Inert (control) group received and inert source plus control antibody. DaRT+aPD-1 group received a 6.5 mm DaRT seed loaded with 80 kBq Ra-224 on day 0 plus anti-PD1 intraperitoneally on days 1, 4, 7, 10, 14 at the dose of 10 mg/kg. GEM group received Gemcitabine (GEM) intraperitoneally on days 0, 3, 7, 10, 14, 17 at the dose of 60 mg/Kg.

On day 14, only the combinational group showed significant reduction in tumor volume compared to the control group. The combinational treatment inhibited tumor development compared to DaRT in the same trend observed for SCC tumors (FIG. 3). Notably, this experiment was terminated on day 20 which is a relatively early timepoint.

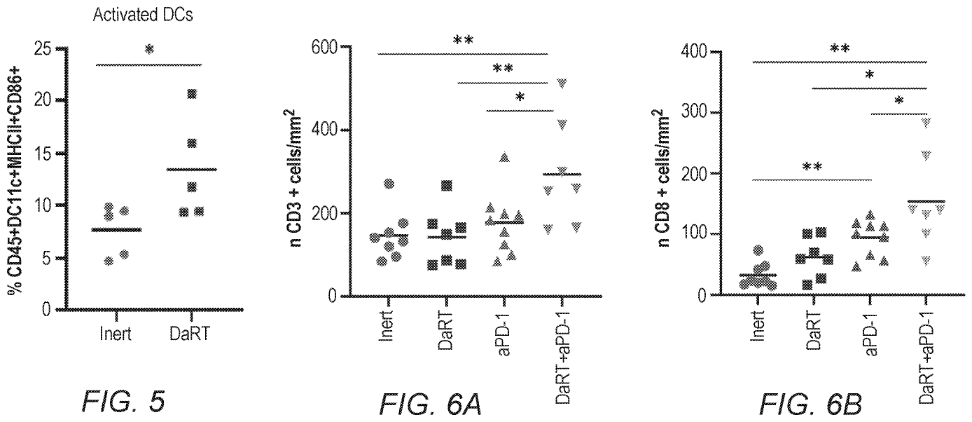

FIG. 5 shows the results of an experiment applicant performed to test the method of FIG. 1. In the experiment, Balb/c mice bearing SCC tumors were treated as follow: aPD-1 group received an inert source plus mouse anti-PD1 intraperitoneally on days 2, 5, 8, 11, 14 at the dose of 10 mg/kg. DaRT group received a 6.5 mm DaRT seed loaded with 75 kBq Ra-224 on day 0 plus control antibody. Inert (control) group received an inert source plus control antibody. DaRT+aPD-1 group received a 6.5 mm DaRT seed loaded with 75 kBq Ra-224 on day 0 plus anti-PD1 was intraperitoneally on days 2, 5, 8, 11, 14 at a dose of 10 mg/kg. In this experiment FACS analysis of the % of intra-tumoral activated Dendritic Cells (DCs) was employed. Tumors were resected at day 7 and enzymatically dissociated with Collagenase (1.5 mg/ml), Hyaluronidase (0.75 mg/ml) and DNase (0.1 mg/ml). The single cell suspensions obtained were incubated for 30 min at 4 °C with the following antibodies mixture: CD11c-PE-cy7, CD86-BV650, CD11b-BB515 (FITC), Ly6G-BV421, Ly6C-PE-CF594 (PI), CD45-APC, MHC Class II-PE. After two washes in FACS Buffer (PBS+2% Fetal Bovine Serum+5 mM EDTA), samples were read in the Stratedigm S1000EXi FACS instrument. Gating strategy: DCs were identified as CD45⁺, CD11c and MHC-II double positive cells. CD86 was stained as an activation marker. The analysis revealed an increase of the % of activated Dendritic Cells (DCs) at an early stage in the DaRT versus the Inert groups. These data suggest that alpha radiation-induced cell death might lead to an enhanced identification of new tumor antigens and due to enhancement in peptide presentation and DAMP signals that results in DCs activation with the subsequent T-cells recruitment and trigger. This supports that DaRT activates the immune system towards a response to the checkpoint blockade.

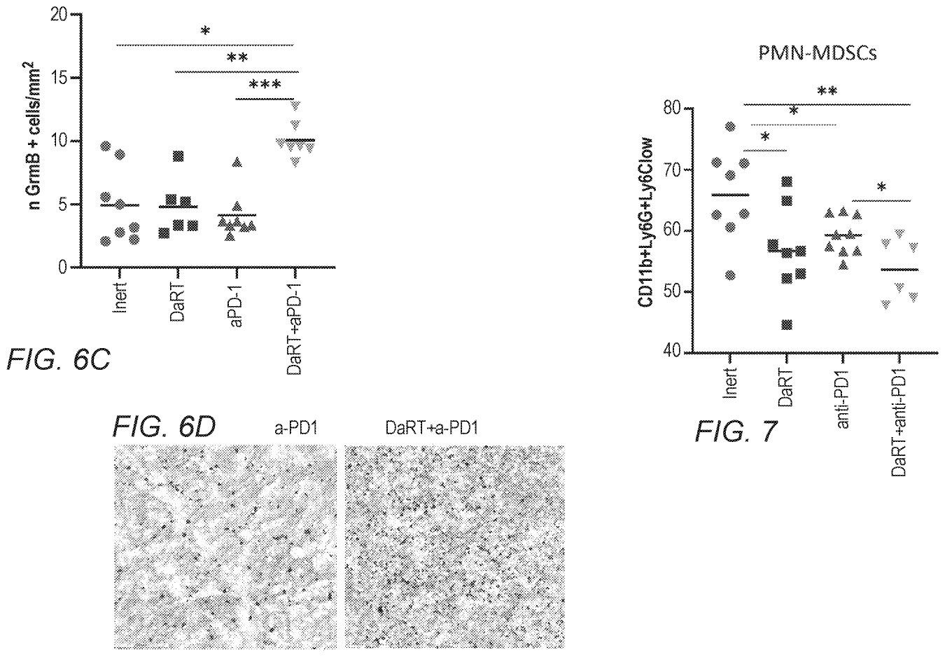

FIG. 6 shows the results of an experiment applicant performed to test the method of FIG. 1. In the experiment, the same treatments were used as described in relation to FIG. 5. The experiment shows the effect of the combination of DaRT with anti-PD1 on T lymphocytes tumor infiltration and functionality, assessed by immunohistochemical staining of the CD3, CD8, and granzyme B molecules. The analysis was done on tumors resected at day 16 post-DaRT insertion (two independent experiments) subjected to immunohistochemistry. Briefly, tumors were frozen in O.C.T. and cryo-sectioned at 5 μm thickness. Tissue sections were then fixed in acetone for 20 minutes, airdried and stained in the Leica Bond III machine. Blocking was done with 5% Normal Goat Serum (NGS), 5% Bovine Serum Albumin (BSA) in PBS for 1 hour. Primary antibodies used were Rabbit anti-mouse CD3 (abcam AB-ab 16669) diluted 1:400, Rabbit anti-mouse CD8 alpha (abcam ab217344) 1:500, Rabbit anti-mouse Granzyme B (abcam ab255598) 1:200, diluted in a Blocking Solution. The secondary antibody was Rabbit-HRP conjugated provided as part of Leica Bond III kit together with DAB Substrate and Hematoxylin. The results clearly show the synergistic effect of DaRT together with the anti-PD1 immune checkpoint inhibitor in increasing the CD3+, CD8+, Granzyme B (FIG. 6A, 6B, 6C, respectively) densities in the tumor. Representative pictures (FIG. 6D) of aPD-1 alone and DaRT+aPD-1 tumors show a clear increase in CD3 T-cells content in the dual therapy. Altogether, these results show a stronger anti-tumor immune response that supports a better therapeutic outcome in the combination therapy when compared to the single treatments.

FIG. 7 shows the results of an experiment applicant performed to test the method of FIG. 1. In the experiment, the same treatments and same FACS staining protocol were used as described in FIG. 5. Gating Strategy: PMN-MDSCs were identified as CD45⁺, CD11b⁺, Ly6G⁺Ly6C^{low} cell populations. FACS analysis on spleens at day 16 post-DaRT insertion from two independent experiments, revealed a decrease in the % of Polymorphonuclear Myeloid Derived Suppressor Cells in DaRT, aPD-1 alone and in the combination (DaRT+anti-PD1) groups when compared to Inert. Interestingly the combined therapy shows a significant decreased in peripheral MDSCs when compared to aPD-1 alone. Since peripheral MDSCs is in correlation with prognosis, this result supports a possible therapeutic advantage in the use of both therapies together.

### CONCLUSION

It will be appreciated that the above-described methods and apparatus are to be interpreted as including apparatus for carrying out the methods and methods of using the apparatus. It should be understood that features and/or steps described with respect to one embodiment may sometimes be used with other embodiments and that not all embodiments of the invention have all of the features and/or steps shown in a particular figure or described with respect to one of the specific embodiments. Tasks are not necessarily performed in the exact order described.

It is noted that some of the above-described embodiments may include structure, acts or details of structures and acts that may not be essential to the invention and which are described as examples. Structure and acts described herein are replaceable by equivalents which perform the same function, even if the structure or acts are different, as known in the art. The embodiments described above are cited by way of example, and the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art. Therefore, the scope of the invention is limited only by the elements and limitations as used in the claims, wherein the terms “comprise,” “include,” “have” and their conjugates, shall mean, when used in the claims, “including but not necessarily limited to.”

1. A method of treating a patient with a tumor, comprising:
treating the tumor with intra-tumoral alpha-emitter radiotherapy; and
administering, to the patient, a checkpoint inhibitor.

2. The method of claim 1, wherein administering the substance comprises administering an anti-LAG3 checkpoint inhibitor.

3. The method of claim 1, wherein administering the substance comprises administering an anti-PD-1 checkpoint inhibitor.

4. The method of claim 1, wherein administering the substance comprises administering an anti-PDL-1 checkpoint inhibitor.

5. The method of claim 1, wherein administering the substance comprises administering an anti-CTLA4 checkpoint inhibitor.

6. The method of claim 1, wherein administering the substance comprises administering an anti-TIGIT checkpoint inhibitor.

7. The method of claim 1, wherein administering the substance comprises administering an anti-TIM-3 checkpoint inhibitor.

8. The method of claim 1, wherein administering the substance comprises administering an anti-CD134 checkpoint inhibitor.

9. The method of claim 1, wherein administering the substance comprises administering an anti-CD137 checkpoint inhibitor.

10. The method of claim 1, wherein administering the substance comprises administering pomalidomide.

11. The method of claim 1, wherein administering the substance comprises administering berzosertib.

12. The method of claim 1, wherein administering the substance comprises administering the substance at least 3 days after beginning the treating of the tumor with intra-tumoral alpha-emitter radiotherapy.

13. The method of claim 1, wherein administering the substance comprises administering the substance at least a week after beginning the treating of the tumor with intra-tumoral alpha-emitter radiotherapy.

14. The method of claim 1, further comprising administering a chemotherapy treatment to the 5 patient.

15. The method of claim 14, wherein the chemotherapy treatment comprises one or more of Cyclophosphamide (CP), doxorubicin, mitoxantrone, gemcitabine, oxaliplatin and/or cisplatin.
